(19) United States
(12) Patent Application Publication
(10) Pub. No.: US 2006/0234360 A1
(43) Pub. Date: Oct. 19, 2006

(54) Ascorbic acid production from D-glucose in yeast

(76) Inventors: Paola Branduardi, Milano (IT); Michael Sauer, Vienna (AT); Diethard Mattanovich, Vienna (AT); Danilo Porro, Erba (IT)

Correspondence Address: WILLIAMS, MORGAN & AMERSON, 10333 RICHMOND, SUITE 1100, HOUSTON, TX, 77042, US

(21) Appl. No.: 11/105,162

(22) Filed: Apr. 13, 2005

(51) Int. Cl. C12P 17/04; C12N 1/18; C12N 15/74

(52) U.S. Cl. 435/126; 435/483; 435/254.2; 435/254.21

(57) ABSTRACT

Herein is disclosed a method of generating ascorbic acid from yeast transformed with a mannose epimerase. In a further embodiment, the yeast can be further transformed with a myoinositol phosphatase. In the method, the transformed yeast can produce L-ascorbic acid from D-glucose. The transformed yeast has been observed to have increased growth rate, cell density, or survival when cultured on appropriate media.

### BACKGROUND OF THE INVENTION

1. Field of the Invention

The present invention relates generally to the field of ascorbic acid production. More particularly, it relates to a process for the production of L-ascorbic acid from yeast, including recombinant yeast.

2. Description of Related Art

L-ascorbic acid (Vitamin C) is a powerful water-soluble antioxidant that is vital for growth and maintenance of all tissue types in humans. One important role of ascorbic acid is its involvement in the production of collagen, an essential cellular component for connective tissues, muscles, tendons, bones, teeth and skin. Collagen is also required for the repair of blood vessels, bruises, and broken bones. Ascorbic acid helps regulate blood pressure, contributes to reduced cholesterol levels, and aids in the removal of cholesterol deposits from arterial walls. Ascorbic acid also aids in the metabolization of folic acid, regulates the uptake of iron, and is required for the conversion of the amino acids L-tyrosine and L-phenylalanine into noradrenaline. The conversion of tryptophan into seratonin, the neurohormone responsible for sleep, pain control, and well-being, also requires adequate supplies of ascorbic acid.

A deficiency of L-ascorbic acid can impair the production of collagen and lead to joint pain, anemia, nervousness and retarded growth. Other effects are reduced immune response and increased susceptibility to infections. The most extreme form of ascorbic acid deficiency is scurvy, a condition evidenced by swelling of the joints, bleeding gums, and the hemorrhaging of capillaries below the surface of the skin. If left untreated, scurvy is fatal.

Although intestines easily absorb ascorbic acid, it is excreted to the urine within two to four hours of ingestion. Therefore, it cannot be stored in the body. L-ascorbic acid is produced in all higher plants and in the liver or kidney of most higher animals, but not humans, bats, some birds and a variety of fishes. Therefore, humans must have access to sufficient amounts of ascorbic acid from adequate dietary sources or supplements in order to maintain optimal health.

Food sources of ascorbic acid include citrus fruits, potatoes, peppers, green leafy vegetables, tomatoes, and berries. Ascorbic acid is also commercially available as a supplement in forms such as pills, tablets, powders, wafers, and syrups.

L-ascorbic acid is approved for use as a dietary supplement and chemical preservative by the U.S. Food and Drug Administration and is on the FDA's list of substances generally recognized as safe. L-ascorbic acid may be used in soft drinks as an antioxidant for flavor ingredients, in meat and meat-containing products, for curing and pickling, in flour to improve baking quality, in beer as a stabilizer, in fats and oils as an antioxidant, and in a wide variety of foods for ascorbic acid enrichment. L-ascorbic acid may also find use in stain removers, hair-care products, plastics manufacture, photography, and water treatment.

The enzymes of the biosynthetic pathway leading to ascorbic acid have not been identified yet to completion. Current understanding of the physiological pathway in plants is shown in FIG. 1.

Two discrete pathways have been reported for ascorbic acid synthesis in plants. In one pathway, L-ascorbic acid is synthesized from D-glucose via L-sorbosone (Loewus M. W. et al., 1990, Plant. Physiol. 94, 1492-1495). Current evidence suggests that the main physiological pathway proceeds from D-glucose via L-galactose and L-galactono-1,4-lactone to L-ascorbic acid (Wheeler G. L. et al. 1998, Nature, 393, 365-369). The last two steps are catalyzed by the enzymes L-galactose dehydrogenase and L-galactono-1,4-lactone dehydrogenase. The last enzyme has been isolated and characterized, and the gene from Brassica oleracea has been cloned and sequenced (Østergaard J. et al. 1997, J. Biol. Chem., 272, 30009-30016).

For use as a dietary supplement, ascorbic acid can be isolated from natural sources or synthesized chemically by the oxidation of L-sorbose as in variations of the Reichstein process (U.S. Pat. No. 2,265,121).

It remains desirable to have methods for the production of ascorbic acid by convenient processes. The synthesis should be enantioselective, because only the L-enantiomer of ascorbic acid is biologically active.

One possible approach is the production of L-ascorbic acid from microorganisms. Microorganisms can be easily grown on an industrial scale. Although the production of L-ascorbic acid from microorganisms and fungi has been reported in the past, recent evidence proves that L-ascorbic acid analogues, and not L-ascorbic acid, were found (Huh W. K. et al. 1998, Mol. Microbiol. 30, 4, 895-903, Hancock R. D. et al., 2000, FEMS Microbiol. Let. 186, 245-250, Dumbrava V. A. et al. 1987, BBA 926, 331-338, Nick J. A. et al., 1986, Plant Science, 46, 181-187). In yeasts (Candida and Saccharomyces species), the production of erythroascorbic acid has been reported (Huh W. K. et al., 1994, Eur. J. Biochem, 225, 1073-1079, Huh W. K. et al., 1998, Mol. Microbiol. 30, 4, 895-903). In such yeasts, a physiological pathway has been proposed proceeding from D-glucose via D-arabinose and D-arabinono-1,4-lactone to erythroascorbic acid (Kim S. T. et al., 1996, BBA, 1297, 1-8). The enzymes D-arabinose dehydrogenase and D-arabinono-1,4-lactone oxidase from Candida albicans as well as S. cerevisiae have been characterized. Interestingly, L-galactose and L-galactono-1,4-lactone are substrates for these activities in vitro.

In vivo production of L-ascorbic acid has been obtained by feeding L-galactono-1,4-lactone to wild-type Candida cells (International Patent Application WO85/01745). Recently it has been shown that wild-type S. cerevisiae cells accumulated intracellularly L-ascorbic acid when incubated with L-galactose, L-galactono-1,4-lactone, or L-gulono-1,4-lactone (Hancock et al., 2000, FEMS Microbiol. Lett. 186, 245-250, Spickett C. M. et al., 2000, Free Rad. Biol. Med. 28, 183-192).

Wild-type Candida cells incubated with L-galactono-1,4-lactone accumulate L-ascorbic acid in the medium, suggesting that this yeast has a biological mechanism for the release of the intracellular accumulated L-ascorbic acid; indeed, L-ascorbic acid is a complex molecule and it is reasonable that its accumulation in the medium is not related to a simple diffusion process, but should depend on facilitated or active transport. This conclusion is supported by the identification and characterization of L-ascorbic acid transporters in higher eukaryotic (mammalian) cells (Daruwala R. et al., 1999, FEBS Letters. 460, 480-484). However, L-ascorbate transporters have not been described among the yeast genera. Nevertheless, while Candida cells growing in media containing L-galactono-1,4-lactone accumulate L-ascorbic acid in the medium, accumulation in the medium of L-ascorbic acid from wild-type S. cerevisiae cells has, surprisingly, never been described.

A desirable method for the large-scale production of ascorbic acid comprises the use of genetically engineered microorganisms (i.e., recombinant microorganisms). Both prokaryotic and eukaryotic microorganisms are today easily and successfully used for the production of heterologous proteins as well as for the production of heterologous metabolites. Among prokaryotes, Escherichia coli and Bacillus subtilis are often used. Among eukaryotes, the yeasts S. cerevisiae and Kluyveromyces lactis are often used.

Østergaard et al. cloned the gene encoding L-galactono-1,4-lactone dehydrogenase from cauliflower in the yeast S. cerevisiae (J. Biol. Chem., 1997, 272, 48, 30009-30016). While, in vitro, the authors found L-galactono-1,4-lactone dehydrogenase activity in the yeast cell extract (cytochrome c assay, see Østergaard et al.), no production of L-ascorbic acid was proven in vivo.

Berry et al., International Patent Appln. WO 99/64618 discuss the potential use of the plant biosynthetic pathway of ascorbic acid; special emphasis is given to the activity catalyzing the conversion of GDP-D-mannose to GDP-L-galactose. However, characterization of the enzyme catalyzing this step has not been presented in detail. An overexpressed E. coli homologue turned out to be inactive.

Smirnoff et al., WO 99/33995, discuss the use of L-galactose dehydrogenase for production of ascorbic acid. The enzyme was purified from pea seedlings and the N-terminal protein sequence was determined.

Roland et al., U.S. Pat. Nos. 4,595,659 and 4,916,068, discuss the use of non-recombinant Candida strains to convert L-galactonic substrates to L-ascorbic acid. Roland et al. described the responsible enzyme as L-galactono-1,4-lactone oxidase.

Kumar, WO 00/34502, discusses the production of L-ascorbic acid in Candida blankii and Cryptococcus dimennae yeast capable of using 2-keto-L-gulonic acid as a sole carbon source in the production. Kumar specifically excludes the production from yeast by a pathway involving L-galactonolactone oxidase or by conversion of L-galactonic precursors.

Previously, we have reported the production of L-ascorbic acid by S. cerevisiae transformed with, inter alia, L-galactose dehydrogenase (LGDH), D-arabinono-1,4-lactone oxidase (ALO), or both and grown in a medium containing one or more of L-galactono-1,4-lactone, L-gulono-1,4-lactone, or L-galactose (U.S. Pat. No. 6,630,330).

It remains desirable to have methods for the production of ascorbic acid by a convenient fermentation process. It would also be desirable to have methods for the production of L-ascorbic acid starting from D-glucose.

### SUMMARY OF THE INVENTION

In one embodiment, the present invention relates to a method of generating L-ascorbic acid, comprising:

a) obtaining a recombinant yeast functionally transformed with a coding region encoding a mannose epimerase (ME);

b) culturing the recombinant yeast in a medium comprising D-glucose, thereby forming L-ascorbic acid, and

c) isolating the L-ascorbic acid.

In another embodiment, the present invention relates to a recombinant yeast functionally transformed with a coding region encoding a mannose epimerase (ME). In further embodiments of the method and the recombinant yeast, the yeast can further be functionally transformed with a coding region encoding a myoinositol phosphatase (MIP).

The present invention provides methods for the production of L-ascorbic acid from D-glucose by a convenient fermentation process.

### DESCRIPTION OF THE DRAWINGS

FIG. 1 shows the main plant pathway for the synthesis of L-ascorbic acid from D-glucose.

FIG. 2 shows the optical density at 660 nm of BY4742 and YML007w yeast in the absence (FIG. 2A) and presence (FIGS. 2B-2C) of oxidative stress. Yap1p activates genes required for the response to oxidative stress; deletion of this gene leads to the observed phenotype

FIG. 3 shows the optical density at 660 nm of BY4742 wt and YML007w yeasts in the presence of oxidative stress and the addition of ascorbic acid to the medium (FIGS. 3A-3B).

FIG. 4 shows the optical density at 660 nm of BY4742 wt; YML007w expressing ALO, LDGH and ME; and YML007w expressing ALO, LDGH, ME and MIP yeasts in the presence of oxidative stress (FIGS. 4A-4B).

FIG. 5 shows the optical density at 660 nm of wild type GRFc; GRF18U expressing ALO, LDGH and ME; and GRF18U expressing ALO, LDGH, ME and MIP yeast strains in the absence (FIG. 5A) and presence (2 mM of H₂O₂) of oxidative stress.

### DESCRIPTION OF ILLUSTRATIVE EMBODIMENTS

In one embodiment, the present invention relates to a method of generating L-ascorbic acid, comprising:

a) obtaining a recombinant yeast functionally transformed with a coding region encoding a mannose epimerase (ME);

b) culturing the recombinant yeast in a medium comprising D-glucose, thereby forming L-ascorbic acid, and

c) isolating the L-ascorbic acid.

A “recombinant” yeast is a yeast that contains a nucleic acid sequence not naturally occurring in the yeast or an additional copy or copies of an endogenous nucleic acid sequence, wherein the nucleic acid sequence is introduced into the yeast or an ancestor cell thereof by human action. Recombinant DNA techniques are well-known, such as in Sambrook et al., Molecular Genetics: A Laboratory Manual, Cold Spring Harbor Laboratory Press, which provides further information regarding various techniques known in the art and discussed herein. In this embodiment, a coding region of the homologous and/or heterologous gene is isolated from an organism, which possesses the gene. The organism can be a bacterium, a prokaryote, a eukaryote, a microorganism, a fungus, a plant, or an animal.

Genetic material comprising the coding region can be extracted from cells of the organism by any known technique. Thereafter, the coding region can be isolated by any appropriate technique. In one known technique, the coding region is isolated by, first, preparing a genomic DNA library or a cDNA library, and second, identifying the coding region in the genomic DNA library or cDNA library, such as by probing the library with a labeled nucleotide probe selected to be or presumed to be at least partially homologous with the coding region, determining whether expression of the coding region imparts a detectable phenotype to a library microorganism comprising the coding region, or amplifying the desired sequence by PCR. Other known techniques for isolating the coding region can also be used.

The yeast to be transformed can be selected from any known genus and species of yeast. Yeasts are described by N. J. W. Kreger-van Rij, “The Yeasts,” Vol. 1 of Biology of Yeasts, Ch. 2, A. H. Rose and J. S. Harrison, Eds. Academic Press, London, 1987. In one embodiment, the yeast genus can be Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces, among others. In a further embodiment, the yeast can be a Saccharomyces, Zygosaccharomyces, or Kluyveromyces spp. In yet a further embodiment, the yeasts can be S. cerevisiae, Z. bailii, or K. lactis. In still a further embodiment, the yeast is S. cerevisiae strain GRF18U, W3031B, BY4742 (MATα; his3; leu2, lys2; ura3, EuroScarf Accession No. Y10000) or YML007w (BY4742ΔYap1) (MATα; his3; leu2, lys2; ura3, Yap1 EuroScarf Accession No. Y10569); Z. bailii ATCC 60483; or K. lactis PM6-7A.

The recombinant yeast is functionally transformed with a coding region encoding a mannose epimerase (D-mannose:L-galactose epimerase; ME). An ME is any GDP-mannose-3,5-epimerase (5.1.3.18), by which is meant an enzyme that catalyzes the conversion of GDP-mannose to GDP-L-galactose. An exemplary ME is provided as SEQ ID NO:1.

In one embodiment, the ME has at least about 95% identity with SEQ ID NO:1. In a further embodiment, the ME has at least about 98% identity with SEQ ID NO:1. “Identity” can be determined by a sequence alignment performed using the ClustalW program and its default values, namely: DNA Gap Open Penalty=15.0, DNA Gap Extension Penalty=6.66, DNA Matrix=Identity, Protein Gap Open Penalty=10.0, Protein Gap Extension Penalty=0.2, Protein matrix=Gonnet. Identity can be calculated according to the procedure described by the ClustalW documentation: “A pairwise score is calculated for every pair of sequences that are to be aligned. These scores are presented in a table in the results. Pairwise scores are calculated as the number of identities in the best alignment divided by the number of residues compared (gap positions are excluded). Both of these scores are initially calculated as percent identity scores and are converted to distances by dividing by 100 and subtracting from 1.0 to give number of differences per site. We do not correct for multiple substitutions in these initial distances. As the pairwise score is calculated independently of the matrix and gaps chosen, it will always be the same value for a particular pair of sequences.”

In still a further embodiment, the ME has SEQ ID NO:1.

In one embodiment, the recombinant yeast is further functionally transformed with a coding region encoding a myoinositol phosphatase (MIP). An MIP is any myoinositol phosphatase (3.1.3.25), by which is meant an enzyme that catalyzes the conversion of L-Galactose-1P to L-galactose. For example, the enzyme L-galactose-1-phosphatase also catalyzes the conversion of L-Galactose-1P to L-galactose and is an MIP by this definition. In one embodiment, the MIP has the sequence provided as SEQ ID NO:2.

In one embodiment, the MIP has at least about 95% identity with SEQ ID NO:2. In a further embodiment, the MIP has at least about 98% identity with SEQ ID NO:2. In still a further embodiment, the MIP has SEQ ID NO:2.

In one embodiment, recombinant yeast is further transformed with a coding region encoding an enzyme selected from L-galactose dehydrogenase (LGDH), L-galactono-1,4-lactone dehydrogenase (AGD), D-arabinose dehydrogenase (ARA), D-arabinono-1,4-lactone oxidase (ALO), or L-gulono-1,4-lactone oxidase (GLO).

The present invention is not limited to the enzymes of the pathways known for the production of L-ascorbic acid intermediates or L-ascorbate in plants, yeast, or other organisms.

In another embodiment, the present invention relates to a microorganism transformed with ME or both ME and MIP which is stress-resistant or robust. In yet another embodiment, the microorganism can be transformed with LGDH, ALO, ME or LGDH, ALO, ME and MIP. The microorganism can be any microorganism, for example, a bacterium, a yeast or another fungus (such as a filamentous fungus), a cultured animal cell, or a cultured plant cell. In one embodiment, the microorganism is a yeast.

It is generally accepted that stresses can lead to perturbation of microbial (yeast) bioprocesses. Stresses may have cellular (internal or intracellular) origins, environmental (external or extracellular) origins, or both. Classical examples of the internally-originating stresses include protein and metabolite overproduction (in terms of weight/volume) and protein and metabolite overproductivity (in terms of weight/volume per unit time), among others.

Examples of externally-originating stresses include high osmolarity, high salinity, oxidative stress, high or low temperature, high or low pH values, presence of organic acids, presence of toxic compounds, and macro- and micro-nutrient starvation, among others.

Stress is typically caused by stressors (or stimuli). Stressors are negative influences on the cell that require the cell to dedicate more effort to maintain equilibrium than is required in the absence of the stressor. This greater effort can lead to a higher or lower metabolic activity, lower growth rate, lower viability, or lower productitivity, among others. Stressors are agents of a physical, chemical or biological nature that represent a change in the usual intracellular or extracellular conditions for any given life form. It follows that while a specific condition (e.g., a temperature of 65° C.) may be stressful (or even lethal) to a certain species that normally lives at 37° C., it will be optimal for a thermophilic organism.

Regardless of the origin, stresses can have different effects, including higher or lower metabolic activity, lower growth rate, lower viability, or lower productitivity, among others. Effects on the cellular or molecular level can include including damage to DNA, damage to lipids, damage to proteins, damage to membranes, damage to other molecules and macromolecules, generation of reactive oxygen species (ROS), induction of apoptosis (programmed cell death), cellular necrosis, cellular lysis, impairment of cellular integrity, and impairment of cellular viability, among others.

ROS can be generated through both intracellular and extracellular stimuli. The majority of endogenous ROS are produced through leakage of these species from the mitochondrial electron transport chain. In addition, cytosolic enzyme systems, including NADPH oxidases, and by-products of peroxisomal metabolism are also endogenous sources of ROS. Generation of ROS also can occur through exposure to numerous exogenous agents and events including ionizing radiation (IR), UV light, chemotherapeutic drugs, environmental toxins, and hyperthermia. Oxidative damage caused by intracellular ROS can result in DNA base modifications, single- and double-strand breaks, and the formation of apurinic/apyrimidinic lesions, many of which are toxic and/or mutagenic. Therefore, the resulting DNA damage may also be a direct contributor to deleterious biological consequences (Tiffany, B. et al., Nucleic Acids Research, 2004, Vol. 32, No. 12, 3712-3723).

In an industrial process, wherein the organism is used as a means for production, stress on the organism typically leads to lower or zero production of the product, lower or zero productivity, a lower or zero yield of the product, or two or more thereof. Stress is therefore a highly undesirable phenomenon, and techniques for minimizing it would be useful.

As used in regard to this embodiment, “production” means the process of making one or more products by a microorganism. (The microorganism itself can be a product, or a compound generated or modified by the metabolic processes of the microorganism can be a product, for example a protein, an organic acid, a vitamin, or an antibiotic). Production can be quantified at any moment in time after commencement of the process by determining the weight of a product produced per weight or volume of the medium on which the microorganism's growth and survival is maintained, or weight or volume of the microorganism's biomass. “Productivity” means the amount of production, as quantified above, over a given period of time (e.g., a rate such as g/L per hour, mg/L per week, or g/g of biomass per hour). “Yield” means the amount of product produced per the amount of substrate converted into the product.

Stress tolerance, stress resistance, or robustness of the strain, as used herein, intend that the micro-organisms show a better industrial perfomance in production processes. This might manifest as one of the following: a better ability to counteract stress, a decrease of the negative impact of stress on the organism, or on productivity, an increase of growth rate or an increase of cell density, a decrease of the inhibition of productivity, a decrease of cellular mortality (increase of viability), a decrease of growth inhibition, or prevention of cellular inactivity due to the stress condition.

We have observed that yeast transformed with ME or both ME and MIP have greater stress resistance or robustness than yeast not transformed with ME or both ME and MIP. This resistance can be against a number of stressors. This greater stress resistance can manifest as one or more of more rapid growth rate of microorganisms expressing ME or ME+MIP in culture, greater cell density of microorganisms expressing ME or ME+MIP in culture, greater survival of microorganisms expressing ME or ME+MIP in culture, or greater production by microorganisms expressing ME or ME+MIP in culture. A measure for cellular survival is the viability (typically given as the fraction of viable cells in relation to the total number of cells). Viability can be determined as the ability of a given cell to form a colony on an appropriate agar plate (reproductive capacity). Cells can furthermore be regarded as viable if they show metabolic activity or if their cellular membrane is intact. It has to be understood that distinct populations of microorganisms might be described as viable and no direct conclusion from one to the other is possible. For example, a cell that is still metabolically active might not be reproductive (colony forming) anymore. Consequently, different methods can be applied to determine the viability of a culture giving different results. Typical methods include plating on agar plates to determine the number of colony forming units, staining with trypane blue and counting under a microscope, whereby the dead cells turn blue, while the viable cells remain color less, due to an intact membrane. Other methods include flow cytometry, whereby the cells are stained by different compounds including propidium iodide (intact membrane prevents entry), ethidium bromide (metabolically active cells exclude stain), among others.

Though not to be bound by theory, we suggest that greater stress resistance as accomplished by the described embodiments results from an increase in antioxidant levels (specifically, L-ascorbate) in the microorganism imparting greater resistance to oxidative stress. We submit this greater stress resistance makes microorgansims expressing ME or ME+MIP, either as such or transformed with additional coding regions, especially suitable for the production of metabolic products in industrial fermentation.

In another embodiment the production, productivity, or yield of a product produced by a microorganism such as a yeast during fermentation is increased by functionally transforming the microorganism with a coding region encoding a mannose epimerase (ME). In yet another embodiment the viability of a microorganism such as a yeast, as defined by the ability to form a colony, as metabolic acitivity or as membrane integrity, during fermentation is increased by functionally transforming the micro-organism with a coding region encoding a mannose epimerase (ME).

Summarizing, the negative effects of stresses typically encountered during microbial production processes can be reduced or abolished by the present invention. That is, the production, productivity, yield, or viability of the microorganism can be increased by establishment or increase of the L-ascorbic acid content of the micro-organism through expression of the enzymes described above.

Therefore, it is particularly useful to express ME, or ME and one or more of the following enzymes: MIP, ALO, or LGDH in a microorganism, if the microorganism shall be cultivated under the condition of osmotic stress. Osmotic stress is a condition in which the microorganism encounters a difference in osmolarity from the optimal osmolarity defined for the respective microorganism of 250 mOsmol or more, or particularly of 500 mOsmol or more or 750 mOsmol or more. For the yeast S. cerevisiae a condition with an osmolarity greater than 500 mOsmol, or greater than 750 mOsmol, or greater than 1000 mOsmol are stressful.

Furthermore, it is particularly useful to express ME, or ME and one or more of the following enzymes: MIP, ALO, or LGDH in a microorganism, if the microorganism shall be cultivated under the condition of pH stress. pH stress is a condition in which the microorganism encounters a difference in pH value from the optimal pH value for production for the respective micro-organism of more than one, or more than two, or more than three pH units. For the yeast S. cerevisiae the typical optimal pH for performance of bioprocesses is 5. A pH of less than 4, or a pH less than 3, or a pH of less than 2 or a pH of more than 6, or a pH of more than 7, or a pH of more than 8 are stressful and in context of the present invention it appears useful to express the described genes in a yeast such as S. cerevisiae if used as a production host under such pH conditions.

It is also particularly useful to express ME, or ME and one or more of the following enzymes: MIP, ALO, or LGDH in a microorganism, if the microorganism shall be cultivated under the condition of temperature stress. Temperature stress is a condition in which the microorganism encounters a cultivation temperature different the optimal temperature value for growth or production for the respective micro-organism by 2° C. or more, by 5° C. or more or even by 10° C. or more. For the yeast S. cerevisiae a temperature at or above 32° C., at or above 35° C., or at or above 40° C. can be stressful. For the bacterium E. coli a temperature at or above 38° C., at or above 41° C., or at or above 46° C. can be stressful.

Further, it is particularly useful to express ME, or ME and one or more of the following enzymes: MIP, ALO, or LGDH in a microorganism, if the microorganism shall be cultivated under the condition of oxidative stress. Oxidative stress is a general term used to describe the steady state level of oxidative damage in a cell, caused by the reactive oxygen species (ROS). This damage can affect a specific molecule or the entire organism. Reactive oxygen species, such as free radicals and peroxides, represent a class of molecules that are derived from the metabolism of oxygen and exist inherently in all aerobic organisms. Oxidative stress results from an imbalance between formation and neutralization of pro-oxidants. Animal cells, among other examples, can be exposed to significant oxidative stress during standard cultivation conditions. Therefore, it appears particularly useful to express ME, or ME and one or more of the following enzymes: MIP, ALO, or LGDH in animal cells.

It is also particularly useful to express ME, or ME and one or more of the following enzymes: MIP, ALO, or LGDH in a microorganism, if the cultivated microorganism is stressed due to overproduction of a metabolite or a protein. Signs of such a stress condition might be the upregulation of genes related to the UPR (unfolded protein response) as known in the art.

The coding region encoding ME, MIP, or another enzyme can be isolated from any source. In one embodiment, the coding region of ME is isolated from Arabidopsis thaliana. In one embodiment, the coding region of MIP is isolated from A. thaliana. It should be noted that a coding region is “isolated” from an organism if it encodes a protein sequence substantially identical to that of the same protein purified from cells of the organism. For example, in the particular embodiments referred to above, the ME coding region or MIP coding region need not be isolated from the nucleic acids of A. thaliana or produced by one or more generations of replication of nucleic acids extracted from A. thaliana.

Preferably, a coding region encoding a desired enzyme is incorporated into the yeast in such a manner that the desired enzyme is produced in the yeast and is substantially functional. Such a yeast may be referred to herein as being “functionally transformed.”

Once the coding region has been extracted from an organism's nucleic acids or synthesized by chemical means, it can be prepared for transformation into and expression in the yeast. At minimum, this involves the insertion of the coding region into a vector and operable linkage to a promoter found on the vector and active in the yeast. Any vector (integrative, chromosomal or episomal) can be used.

Any promoter active in the target host (homologous or heterologous; constitutive, inducible or repressible) can be used. Such insertion can involve the use of restriction endonucleases to “open up” the vector at a desired point where operable linkage to the promoter is possible, followed by ligation of the coding region into the desired point. If desired, before insertion into the vector, the coding region can be prepared for use in the target organism. This can involve altering the codons used in the coding region to more fully match the codon use of the target organism; changing sequences in the coding region that could impair the transcription or translation of the coding region or the stability of an mRNA transcript of the coding region; or adding or removing portions encoding signaling peptides (regions of the protein encoded by the coding region that direct the protein to specific locations (e.g. an organelle, the membrane of the cell or an organelle, or extracellular secretion)), among other possible preparations known in the art.

Regardless whether the coding region is modified, when the coding region is inserted into the vector, it is operably linked to a promoter active in the yeast. A promoter, as is known, is a DNA sequence that can direct the transcription of a nearby coding region. As already described, the promoter can be constitutive, inducible or repressible. Constitutive promoters continually direct the transcription of a nearby coding region. Inducible promoters can be induced by the addition to the medium of an appropriate inducer molecule, which will be determined by the identity of the promoter. Repressible promoters can be repressed by the addition to the medium of an appropriate repressor molecule, which will be determined by the identity of the promoter. In one embodiment, the promoter is constitutive. In a further embodiment, the constitutive promoter is the S. cerevisiae triosephosphateisomerase (TPI) promoter.

The vector comprising the coding region operably linked to the promoter can be a plasmid, a cosmid, or a yeast artificial chromosome, among others known in the art to be appropriate for use in yeast. In addition to the coding region operably linked to the promoter, the vector can also comprise other genetic elements. For example, if the vector is not expected to integrate into the yeast genome, the vector can comprise an origin of replication, which allows the vector to be passed on to progeny cells of a yeast comprising the vector. If integration of the vector into the yeast genome is desired, the vector can comprise sequences homologous to sequences found in the yeast genome, and can also comprise coding regions that can facilitate integration. To determine which yeast cells are transformed, the vector can comprise a selectable marker or screenable marker which imparts a phenotype to the yeast that distinguishes it from untransformed yeast, e.g. it survives on a medium comprising an antibiotic fatal to untransformed yeast or it metabolizes a component of the medium into a product that the untransformed yeast does not, among other phenotypes. In addition, the vector may comprise other genetic elements, such as restriction endonuclease sites and others typically found in vectors.

After the vector is prepared, with the coding region operably linked to the promoter, the yeast can be transformed with the vector (i.e. the vector can be introduced into at least one of the cells of a yeast population). Techniques for yeast transformation are well established, and include electroporation, microprojectile bombardment, and the LiAc/ssDNA/PEG method, among others. Yeast cells, which are transformed, can then be detected by the use of a screenable or selectable marker on the vector. It should be noted that the phrase “transformed yeast” has essentially the same meaning as “recombinant yeast,” as defined above. The transformed yeast can be one that received the vector in a transformation technique, or can be a progeny of such a yeast.

After a recombinant yeast has been obtained, the yeast can be cultured in a medium. The medium in which the yeast can be cultured can be any medium known in the art to be suitable for this purpose. Culturing techniques and media are well known in the art. In one embodiment, culturing can be performed by aqueous fermentation in an appropriate vessel. Examples for a typical vessel for yeast fermentation comprise a shake flask or a bioreactor.

The medium can comprise D-glucose. It can further comprise any other component required for the growth of the yeast. D-glucose can be a component required for the growth of the yeast but need not be.

The medium can comprise a carbon source other than D-glucose, such as sucrose, fructose, lactose, D-galactose, or hydrolysates of vegetable matter, among others. In one embodiment, the medium can also comprise a nitrogen source as either an organic or an inorganic molecule. In a further embodiment, the medium can also comprise components such as amino acids; purines; pyrimidines; corn steep liquor; yeast extract; protein hydrolysates; water-soluble vitamins, such as B complex vitamins; or inorganic salts such as chlorides, hydrochlorides, phosphates, or sulfates of Ca, Mg, Na, K, Fe, Ni, Co, Cu, Mn, Mo, or Zn, among others. Further components known to one of ordinary skill in the art to be useful in yeast culturing or fermentation can also be included. The medium can be buffered but need not be.

During the course of the fermentation, the D-glucose is internalized by the yeast and converted, through a number of steps, into L-ascorbic acid. The L-ascorbic acid so produced can be collected within the yeast, or can be secreted by the yeast into the medium.

A preferred medium comprises D-glucose and YNB.

After culturing has progressed for a sufficient length of time to produce a desired concentration of L-ascorbic acid in the yeast, the medium, or both, the L-ascorbic acid can be isolated. “Isolated,” as used herein to refer to ascorbic acid, means being brought to a state of greater purity by separation of L-ascorbic acid from at least one non-L-ascorbic acid component of the yeast or the medium. Preferably, the isolated L-ascorbic acid is at least about 95% pure, more preferably at least about 99% pure.

To isolate L-ascorbic acid from the yeast, the first step of isolation, after the yeast is separated from the medium, can be lysing of the yeast by chemical or enzymatic treatment, treatment with glass beads, sonication, freeze/thaw cycling, or other known techniques. L-ascorbic acid can be purified from the membrane, protein, and nucleic acid fractions of the yeast lysate by appropriate techniques, such as centrifugation, filtration, microfiltration, ultrafiltration, nanofiltration, liquid-liquid extraction, crystallization, enzymatic treatment with nuclease or protease, or chromatography, among others.

To isolate L-ascorbic acid accumulated in the medium, the isolation can comprise purifying the ascorbic acid from the medium. Purification can be performed by known techniques, such as the use of an ion exchange resin, activated carbon, microfiltration, ultrafiltration, nanofiltration, liquid-liquid extraction, crystallization, or chromatography, among others.

L-ascorbic acid can be isolated from both the yeast and the medium.

If the yeast accumulates L-ascorbic acid in the medium during the culturing step, preferably the concentration of L-ascorbic acid is stabilized or allowed to increase.

The Following Definitions are Provided in Order to Aid Those Skilled in the Art in Understanding the Detailed Description of the Present Invention.

The term “accumulation of ascorbic acid above background levels” refers to the accumulation of ascorbic acid above the undetectable levels as determined using the procedures described herein.

“Ascorbic acid” as well as “ascorbate” as used herein, refers to L-ascorbic acid.

“Ascorbic acid precursor” is a compound that can be converted by a yeast of the present invention, either directly or through one or more intermediates, into L-ascorbic acid.

“Amplification” refers to increasing the number of copies of a desired nucleic acid molecule or to increase the activity of an enzyme, by whatsoever means.

“Codon” refers to a sequence of three nucleotides that specify a particular amino acid.

“DNA ligase” refers to an enzyme that covalently joins two pieces of double-stranded DNA.

“Electroporation” refers to a method of introducing foreign DNA into cells that uses a brief, high voltage DC charge to permeabilize the host cells, causing them to take up extra-chromosomal DNA.

“Endonuclease” refers to an enzyme that hydrolyzes double stranded DNA at internal locations.

Enzyme 1.1.3.37, D-arabinono-1,4-lactone oxidase, refers to a protein that catalyzes the conversion of D-arabinono-1,4-lactone+O₂ to D-erythroascorbate+H₂O₂. The same enzyme due to broadness of substrate range catalyses the conversion of L-galactono-1,4-lactone+O₂ to L-ascorbic acid+H₂O₂. Erroneously the same enzyme is referred to as L-galactono-1,4-lactone oxidase (enzyme 1.1.3.24) (see Huh, W. K. et al, 1998, Mol. Microbiol. 30, 4, 895-903)

Enzyme 1.3.2.3, L-galactono-1,4-lactone dehydrogenase, refers to a protein that catalyzes the conversion of L-galactono-1,4-lactone+2 ferricytochrome C to L-ascorbic acid+2 ferrocytochrome C.

Enzyme 1.1.3.8, L-gulono-1,4-lactone oxidase, refers to a protein that catalyzes the oxidation of L-gulono-1,4-lactone to L-xylo-hexulonolactone which spontaneously isomerizes to L-ascorbic acid.

Enzyme GDP-mannose-3,5-epimerase (5.1.3.18), refers to a protein that catalyzes the conversion of GDP-mannose to GDP-L-galactose.

Enzyme myoinositol phosphatase (3.1.3.23), refers to a protein that catalyzes the conversion of L-Galactose-1P to L-galactose.

Other enzymes of interest, and their classification numbers, are as follows:

| | |
|---|---|
| Hexokinase | 2.7.1.1 |
| Glucose-6-P isomerase | 5.3.1.9 |
| Mannose-6-P isomerase | 5.3.1.8 |
| phosphomannomutase | 5.4.2.8 |
| Mannose-1-P guanylyltransferase | 2.7.7.22 |
| GDP-Mannose 3,5-epimerase | 5.1.3.18 |
| Sugar phosphatase | 3.1.3.23 |
| L-Galactose-dehydrogenase | *) |
| L-Galactono-1,4-lactone dehydrogenase | 1.3.2.3 |
| D-Mannose kinase | 2.7.1.1 |
| Phosphoglucomutase | 5.4.2.2 |
| UTP-Glucose-1-P uridylyl transferase | 2.7.7.9 |
| UDP-D-Glucose dehydrogenase | 1.1.1.22 |
| UDP-Glucuronate 4-epimerase | 5.1.3.6 |
| glucuronate-1-P uridylyltransferase | 2.7.7.44 |
| D-Glucuronokinase | 2.7.1.43 |
| D-Glucuronate reductase | 1.1.1.19 |
| Aldonolactonase | 3.1.1.17 |
| L-Gulono-1,4-lactone oxidase | 1.1.3.8 |
| Uronolactonase | 3.1.1.19 |
| Glucuronolactone reductase activity | 1.1.1.20 |
| L-Galactono-1,4-lactone 3-epimerase | *) |
| Galacturonate-1-P uridylyltransferase | *) |
| Galacturonokinase | 2.7.1.44 |
| Hexuronate (D-galacturonate) reductase | *) |
| Myoinositol 1-P synthase | 5.5.1.4 |
| Myoinositol 1-P monophosphatase | 3.1.3.25 |
| Myoinositol oxygenase | 1.13.99.1 |
| D-Galactokinase | 2.7.1.6 |
| UTP-Hexose 1-P uridylyltransferase | 2.7.7.10 |
| UDP-Glucose 4-epimerase | 5.1.3.2 |
| Suc synthase | 2.4.1.13 |
| Fructokinase | 2.7.1.4 |

*) Classification number not available in databases.

The term “expression” refers to the transcription of a gene to produce the corresponding mRNA and translation of this mRNA to produce the corresponding gene product, i.e., a peptide, polypeptide, or protein.

The phrase “functionally linked” or “operably linked” refers to a promoter or promoter region and a coding or structural sequence in such an orientation and distance that transcription of the coding or structural sequence may be directed by the promoter or promoter region.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

The term “genome” encompasses both the chromosomes and plasmids within a host cell. Encoding DNAs of the present invention introduced into host cells can therefore be either chromosomally integrated or plasmid-localized.

“Heterologous DNA” refers to DNA from a source different than that of the recipient cell.

“Homologous DNA” refers to DNA from the same source as that of the recipient cell.

“Hybridization” refers to the ability of a strand of nucleic acid to join with a complementary strand via base pairing. Hybridization occurs when complementary sequences in the two nucleic acid strands bind to one another.

The term “medium” refers to the chemical environment of the yeast comprising any component required for the growth of the yeast or the recombinant yeast and one or more precursors for the production of ascorbic acid. Components for growth of the yeast and precursors for the production of ascorbic acid may or may be not identical.

“Open reading frame (ORF)” refers to a region of DNA or RNA encoding a peptide, polypeptide, or protein.

“Plasmid” refers to a circular, extra chromosomal, replicatable piece of DNA.

“Polymerase chain reaction (PCR)” refers to an enzymatic technique to create multiple copies of one sequence of nucleic acid. Copies of DNA sequence are prepared by shuttling a DNA polymerase between two amplimers. The basis of this amplification method is multiple cycles of temperature changes to denature, then re-anneal amplimers, followed by extension to synthesize new DNA strands in the region located between the flanking amplimers.

The term “promoter” or “promoter region” refers to a DNA sequence, usually found upstream (5′) to a coding sequence, that controls expression of the coding sequence by controlling production of messenger RNA (mRNA) by providing the recognition site for RNA polymerase and/or other factors necessary for start of transcription at the correct site.

A “recombinant cell” or “transformed cell” is a cell that contains a nucleic acid sequence not naturally occurring in the cell or an additional copy or copies of an endogenous nucleic acid sequence, wherein the nucleic acid sequence is introduced into the cell or an ancestor thereof by human action.

The term “recombinant vector” or “recombinant DNA or RNA construct” refers to any agent such as a plasmid, cosmid, virus, autonomously replicating sequence, phage, or linear or circular single-stranded or double-stranded DNA or RNA nucleotide sequence, derived from any source, capable of genomic integration or autonomous replication, comprising a nucleic acid molecule in which one or more sequences have been linked in a functionally operative manner. Such recombinant constructs or vectors are capable of introducing a 5′ regulatory sequence or promoter region and a DNA sequence for a selected gene product into a cell in such a manner that the DNA sequence is transcribed into a functional mRNA, which may or may not be translated and therefore expressed.

“Restriction enzyme” refers to an enzyme that recognizes a specific sequence of nucleotides in double stranded DNA and cleaves both strands; also called a restriction endonuclease. Cleavage typically occurs within the restriction site or close to it.

“Selectable marker” refers to a nucleic acid sequence whose expression confers a phenotype facilitating identification of cells containing the nucleic acid sequence. Selectable markers include those, which confer resistance to toxic chemicals (e.g. ampicillin, kanamycin) or complement a nutritional deficiency (e.g. uracil, histidine, leucine).

“Screenable marker” refers to a nucleic acid sequence whose expression imparts a visually distinguishing characteristic (e.g. color changes, fluorescence).

“Transcription” refers to the process of producing an RNA copy from a DNA template.

“Transformation” refers to a process of introducing an exogenous nucleic acid sequence (e.g., a vector, plasmid, or recombinant nucleic acid molecule) into a cell in which that exogenous nucleic acid is incorporated into a chromosome or is capable of autonomous replication. A cell that has undergone transformation, or a descendant of such a cell, is “transformed” or “recombinant.” If the exogenous nucleic acid comprises a coding region encoding a desired protein, and the desired protein is produced in the transformed yeast and is substantially functional, such a transformed yeast is “functionally transformed.”

“Translation” refers to the production of protein from messenger RNA.

The term “yield” refers to the amount of ascorbic acid produced (molar or weight/volume) divided by the amount of precursor consumed (molar or weight/volume) multiplied by 100.

“Unit” of enzyme refers to the enzymatic activity and indicates the amount of micromoles of substrate converted per mg of total cell proteins per minute.

“Vector” refers to a DNA or RNA molecule (such as a plasmid, cosmid, bacteriophage, yeast artificial chromosome, or virus, among others) that carries nucleic acid sequences into a host cell. The vector or a portion of it can be inserted into the genome of the host cell.

List of Abbreviations:
Asc L-ascorbic acid (vitamin C)
AGD L-galactono-1,4-lactone dehydrogenase (without signaling peptide)
ALO D-arabinono-1,4-lactone oxidase
ARA D-arabinose dehydrogenase
Gal L-galactono-1,4-lactone
Gul L-gulono-1,4-lactone
LGDH L-galactose dehydrogenase
ME Mannose epimerase
MIP Myoinositol phosphatase
RGLO L-gulono-1,4-lactone oxidase
TCA trichloro acetic acid
TPI triosephosphateisomerase

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Materials and Methods

1. Determination of Ascorbic Acid

Ascorbic acid was determined spectrophotometrically following a method after Sullivan et al. (1955, Assoc. Off. Agr. Chem., 38, 2, 514-518). 135 μl of sample were mixed in a cuvette with 40 μl of H₃PO₄ (85%). Then 675 μl α,α′-Bipyridyl (0.5%) and 135 μl FeCl₃ (1%) were added. After 10 min the absorbance at 525 nm was measured. In some experiments, the identity of the ascorbic acid was confirmed by HPLC (Tracer Extrasil Column C8, 5 μM, 15×0.46 cm, Teknokroma, S. Coop. C. Ltda. #TR-016077; Eluent: 5 mM cetyltrimethylammonium bromide, 50 mM KH₂PO₄ in 95/5 H₂O/Acetonitrile; Flow rate: 1 ml min⁻¹, Detection UV@254 nm) with pure L-ascorbic acid (Aldrich, A9,290-2) as standard.

2. Amplification of Specific Gene Sequences

To amplify specific gene sequences, PfuTurbo DNA polymerase (Stratagene#600252) was used on a GeneAmp PCR System 9700 (PE Appl. Biosystems, Inc.). Standard conditions used were: 400 μM dNTP, 0.5 μM primers, 0.5 mM MgCl₂ (in addition to the buffer), and 3.75 U Pfu per 100 μl reaction.

The sequences of the genes used have been publicly reported via Genbank, as follows, except for MIP. The MIP sequence listed as SEQ ID NO:4 differed from the Genbank sequence, accession no. NM_{—}111155, by two translationally silent point substitutions: at bp271, A (NM_{—}111155) to T (SEQ ID NO:4); at bp 685, T (NM_{—}11155) to G (SEQ ID NO:4).

| Gene | Genbank accession no(s). | SEQ ID NO: |
|---|---|---|
| ME | AY116953 | 3 |
| MIP | n.a. | 4 |
| ALO | U40390, AB009401 | 5, 6 |
| LGDH | | 7 |

The following program was used for amplification of ALO:

| | | | | |
|---|---|---|---|---|
| 94° C. | 5 | min | | |
| 94° C. | 45 | s | | |
| 50° C. | 30 | s | {close oversize brace} | 33 cycles |

| | |
|---|---|
| 72° C. | 1 min 40 s |

| | | |
|---|---|---|
| 72° C. | 7 | min |

| | |
|---|---|
| 4° C. | ∞ |

The following program was used for amplification of LGDH:

| | | | | |
|---|---|---|---|---|
| 94° C. | 5 | min | | |
| 94° C. | 45 | s | | |
| 56° C. | 30 | s | {close oversize brace} | 33 cycles |

| | |
|---|---|
| 72° C. | 1 min 40 s |

| | | |
|---|---|---|
| 72° C. | 7 | min |

| | |
|---|---|
| 4° C. | ∞ |

The following program was used for amplification of ME:

| | | | | |
|---|---|---|---|---|
| 94° C. | 5 | min | | |
| 94° C. | 15 | s | | |
| 50° C. | 30 | s | {close oversize brace} | 30 cycles |

| | |
|---|---|
| 72° C. | 1 min 30 s |

| | | |
|---|---|---|
| 72° C. | 7 | min |

| | |
|---|---|
| 4° C. | ∞ |

The following program was used for amplification of MIP:

| | | | | | |
|---|---|---|---|---|---|
| 94° | C. | 5 | min | | |
| 94° | C. | 15 | s | | |
| 59.8° | C. | 30 | s | {close oversize brace} | 28 cycles |
| 72° | C. | 45 | s | | |
| 72° | C. | 7 | min | | |

| | | |
|---|---|---|
| 4° | C. | ∞ |

Template DNA for LGDH, ME, and MIP: 50 ng plasmid cDNA library pFL61 Arabidopsis (ATCC #77500 (Minet M. et al, 1992, Plant J., 2, 417-422)). Template DNA for ALO: 50 ng genomic DNA from S. cerevisiae GRF18U, extracted using a standard method. PCR products were blunt end cloned into the EcoRV site of pSTBlue-1 using the perfectly blunt cloning kit from Novagen Inc. (#70191-4).

| | |
|---|---|
| Oligonucleotides used | Gene amplified |

| | |
|---|---|
| SEQ ID NO:8: tttcaccatatgtctactatcc | ALO (yeast) |
| SEQ ID NO:9: aaggatcctagtcggacaactc | |
| SEQ ID NO:10: atgacgaaaatagagcttcgagc | LGDH (plant) |
| SEQ ID NO:11: ttagttctgatggattccacttgg | |
| SEQ ID NO:12: gcgccatgggaactaccaatggaaca | ME (plant) |
| SEQ ID NO:13: gcgctcgagtcactcttttccatca | |
| SEQ ID NO:14: atccatggcggacaatgattctc | MIP (plant) |
| SEQ ID NO:15: aatcatgcccctgtaagccgc | |

3. Plasmid Construction

The naming convention used herein is that pSTBlue-1 containing, for example, ALO in the sense direction regarding its multiple cloning site (MCS) was designated pSTB ALO-1. In a further example, pSTBlue-1 containing ALO in the antisense direction regarding its MCS was designated pSTB ALO-2, and so on.

Inserts were cloned using either the pYX series (R&D Systems, Inc.) or the centromeric expression plasmids pZ₃ and pZ₄ (P. Branduardi, M. Valli, L. Brambilla, M. Sauer, L. Alberghina and D. Porro. The Yeast Zygosaccharomyces bailii: a New Host for Heterologous Protein Production, Secretion and for Metabolic Engineering Applications, FEBS Yeast Research, FEMS Yeast Res. 4, 493-504, 2004). Standard procedures were employed for all cloning purposes (Sambrook J. et al., Molecular Genetics: A Laboratory Manual, Cold Spring Harbor Laboratory Press).

| | | | | |
|---|---|---|---|---|
| pSTB LGDH-1 | EcoRI | pYX022 | pH LGDH | HIS 3 (marker) |
| pSTB ALO-1 | EcoRI | pYX042 | pL ALO | LEU2 (marker) |
| pSTB ME-1 | EcoRI | pZ₃ | pZ₃ ME | Kan^{r} (marker) |
| pSTB ME-1 | EcoRI | pZ₄ | PZ₄ ME | Hph^{r} (marker) |
| pSTB MIP-1 | EcoRI | pYX012 | pU MIP | URA 3 (marker) |

For all the work performed below, the yeast control strains were transformed with the corresponding empty vectors.

4. Yeast Cultivation and Examination:

Yeast strains used were S. cerevisiae GRF18U (Brambilla, L. et al., 1999, FEMS Microb. Lett. 171, 133-140), S. cerevisiae GRFc (Brambilla et al. 1999 FEMS Microb. Lett. 171: 133-140), S. cerevisiae BY4742 (MATα; his3; leu2, lys2; ura3, EuroScarf Accession No. Y10000), S. cerevisiae YML007w (BY4742; MATα, his3, leu2, lys2, ura3 YML007w::KanMX4, EuroScarf Accession No. Y10569), or derived from them through transformation with the different developed plasmids. All strains were cultivated in shake flasks in minimal medium (0.67% w/v YNB (Difco Laboratories, Detroit, Mich. #919-15), 2% w/v glucose or mannose, with addition of the appropriate amino acids or adenine or uracil, respectively, to 50 μg L-¹) and/or the appropriate antibiotic (G418 or hygromicin to 500 mg/l and 400 mg/l, respectively) under standard conditions (shaking at 30° C.). The initial optical density at 660 nm was about 0.05 for ascorbic acid determination, and 0.1 for the kinetics of the recovery from oxidative stress.

Cells were recovered by centrifugation at 4000 rpm for 5 min at 4° C., washed once with cold distilled H₂O, and treated as follows: for determination of intracellular ascorbic acid, cells were resuspended in about 3 times the pellet volume of cold 10% TCA, vortexed vigorously, kept on ice for about 20 min, and then the supernatant was cleared from the cell debris by centrifugation.

5. Yeast Transformation:

Transformation of yeast cells was done following the standard LiAc/ss-DNA/PEG method (Gietz, R. D. and Schiestl, R. H., 1996, Transforming Yeast with DNA, Methods in Mol. and Cell. Biol.). Transformed yeast are being deposited with ATCC, catalog numbers not yet assigned.

Experimental Results

Expression of Arabidopsis thaliana ME, MIP, LDGH and S. cerevisiae ALO in GRF18U

The genes encoding A. thaliana ME, S. cerevisiae ALO, A. thaliana LGDH, and A. thaliana MIP were placed under the control of the TPI promoter each on its own integrative plasmid, except ME, which was sub-cloned in a centromeric plasmid. Two or more of the genes were integrated into S. cerevisiae GRF18U and BY4742. Each gene was integrated at a unique locus.

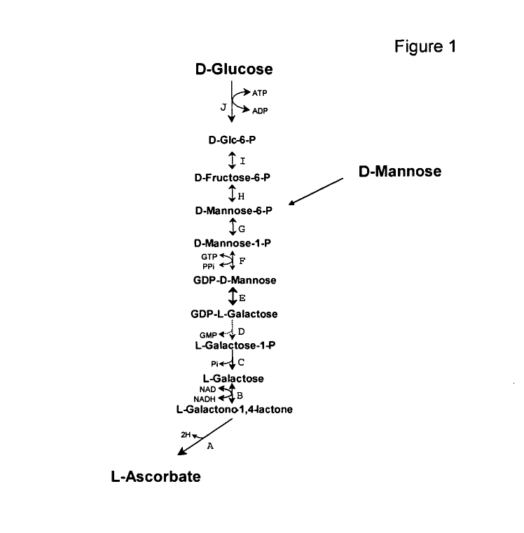

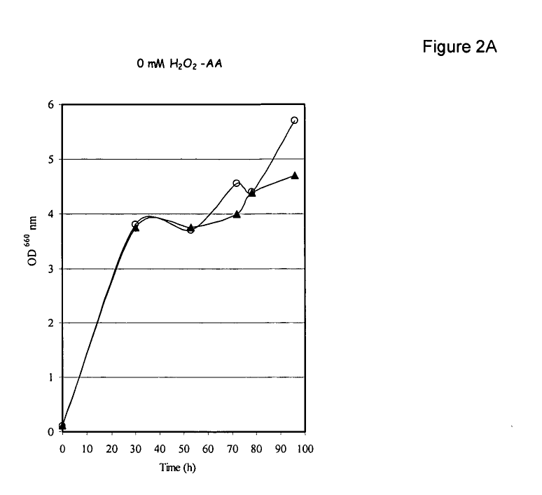

FIG. 1 provides a schematic representation of the current understanding of the physiological biosynthetic pathway leading from D-glucose to L-ascorbic acid in plants. The following enzymes are involved: A, L-galactono-1,4-lactone dehydrogenase (1.3.2.3), B, L-galactose dehydrogenase, C, myoinositol phosphatase (3.1.3.23), D, hydrolase (putative), E, GDP-mannose-3,5-epimerase (5.1.3.18), F, mannose-1-phosphate guanylyltransferase (2.7.7.22), G, phosphomannomutase (5.4.2.8), H, mannose-6-phosphate isomerase (5.3.1.8), I, glucose-6-phosphate isomerase (5.3.1.9), J; hexokinase (2.7.1.1).

In the pathway shown in FIG. 1, ALO catalyzes reaction A, LGDH catalyzes reaction B, ME catalyzes reaction E, and MIP catalyzes reaction C.

Wild-type yeast cells are known to produce GDP-mannose (reactions F-J in FIG. 1) and to transport it to the endoplasmic reticulum.

The table below shows the conversion of D-Glucose and D-Mannose to ascorbic acid by S. cerevisiae GRFc (control), or S. cerevisiae GRF18U transformed with (i) ALO and LDGH; (ii) ALO, LDGH and ME; or (iii) ALO, LDGH, ME and MIP. Cells were grown on mineral medium (2% glucose or mannose, 0.67% YNB) starting from an OD⁶⁶⁰ of 0.05. After 24 hours of growth, ascorbic acid was determined. While both the wild-type GRFc and GRF18U cells transformed with ALO and LGDH did not accumulate ascorbic acid, cells transformed with ALO, LDGH and ME, or ALO, LDGH, ME and MIP, respectively unexpectedly accumulated considerable amounts (i.e. greater than background levels) of ascorbic acid.

Transformed yeast were batch grown on glucose- or mannose-based media:

| | Total (ascorbate | Total (ascorbate plus |
|---|---|---|
| | plus erythroascorbate) | erythroascorbate) on |
| | on glucose-containing | mannose-containing |
| Expressed gene | media | media |
| Wt (control) | 0.0205 | 0.0220 |
| ALO, LGDH (control) | 0.0210 | 0.0221 |
| ALO, LDGH, ME | 0.0302 | 0.0332 |
| ALO, LDGH, ME, MIP | 0.0450 | 0.0296 |

(Total (ascorbate plus erythroascorbate) values are mg/OD⁶⁶⁰ of Biomass/L)

The values determined in the control strain indicate the production of erythroascorbate normally produced by wild type yeasts.

We conclude that the yeast endogenously possesses activities which can nonspecifically catalyze reactions from GDP-L-galactose to L-galactose (see FIG. 1). Specifically, though not to be bound by theory, we conclude that GDP-L-galactose spontaneously hydrolyses to L-galactose-1-P and that a nonspecific phosphatase catalyzed the conversion of L-galactose-1-P to L-galactose, which was then converted to L-ascorbic acid by LGDH and ALO. MIP provided superior catalysis of L-galactose-1-P to L-galactose than did the putative nonspecific phosphatase (ALO, LGDH, ME, MIP vs. ALO, LGDH, ME).

We did not observe any ascorbic acid accumulation in the medium.

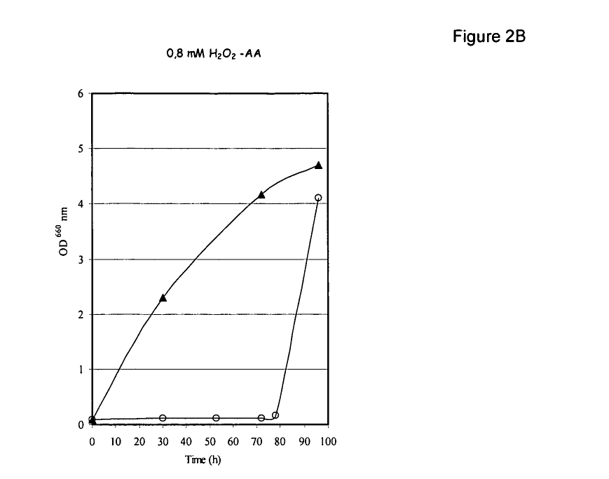

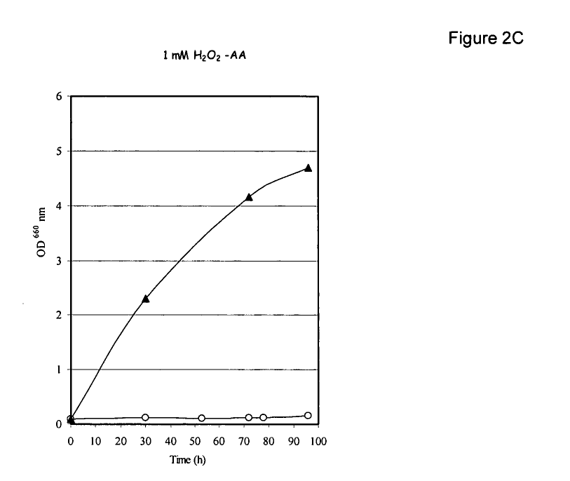

FIG. 2 shows that YML007w yeast hosts are particularly sensitive to oxidative stress. Yap1p activates genes required for the response to oxidative stress; deletion of this gene leads to the observed phenotype (Rodrigues-Pousada C A, Nevitt T, Menezes R, Azevedo D, Pereira J, Amaral C. Yeast activator proteins and stress response: an overview. FEBS Lett. Jun. 1, 2004; 567(1):80-85)

The following yeast strains have been analyzed:

BY4742

[symbol]

YML007

[symbol]

FIG. 2A. The yeast strains were grown on mineral medium (2% glucose, 0.67% YNB) starting from an OD⁶⁶⁰ of 0.1.

FIG. 2B. The yeast strains were grown on mineral medium (2% glucose, 0.67% YNB) starting from an OD⁶⁶⁰ of 0.1 in the presence of 0.8 mM of H₂O₂.

FIG. 2C. The yeast strains were grown on mineral medium (2% glucose, 0.67% YNB) starting from an OD⁶⁶⁰ of 0.1 in the presence of 1.0 mM of H₂O₂.

The two strains grew in absence of H₂O₂ (FIG. 2A) while growth of the YML007w yeast host is strongly delayed in medium containing 0.8 mM of hydrogen peroxide (FIG. 2B) and completely impaired in the medium containing 1 mM of hydrogen peroxide (FIG. 2C).

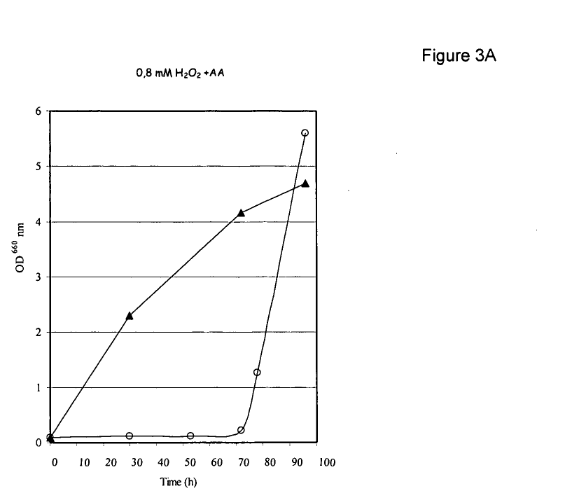

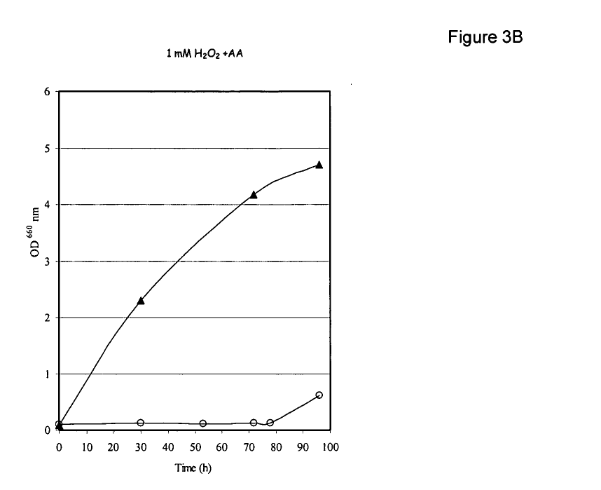

FIG. 3 shows that the growth sensitivity of YML007w yeasts can be rescued by adding ascorbic acid to the medium.

The following yeast strains have been analyzed:

BY4742

[symbol]

YML007w

[symbol]

FIG. 3A. The yeast strains were grown on mineral medium (2% glucose, 0.67% YNB) starting from an OD⁶⁶⁰ of 0.1 in presence of 0.8 mM of H₂O₂. Ascorbic acid was added at T=0 at a final concentration of 15 mg/L.

FIG. 3B. The yeast strains were grown on mineral medium (2% glucose, 0.67% YNB) starting from an OD⁶⁶⁰ of 0.1 in presence of 1.0 mM of H₂O₂. Ascorbic acid was added at T=0 at a final concentration of 15 mg/L.

The effect of added ascorbic acid is concentration dependent. In fact, an increase of the ascorbic acid concentration to 30 mg/L, determined a faster rescue of the growth defects (data not shown in figure).

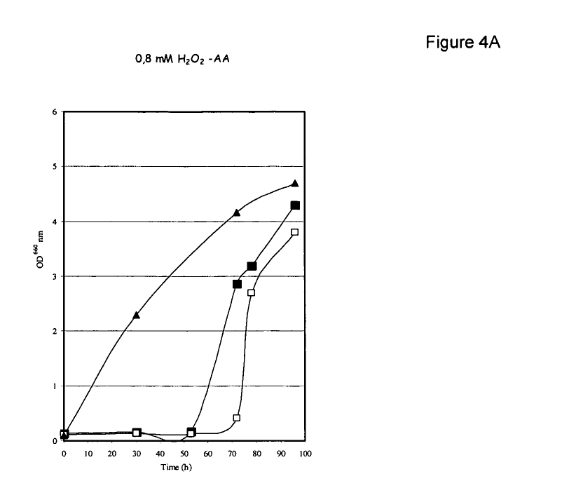

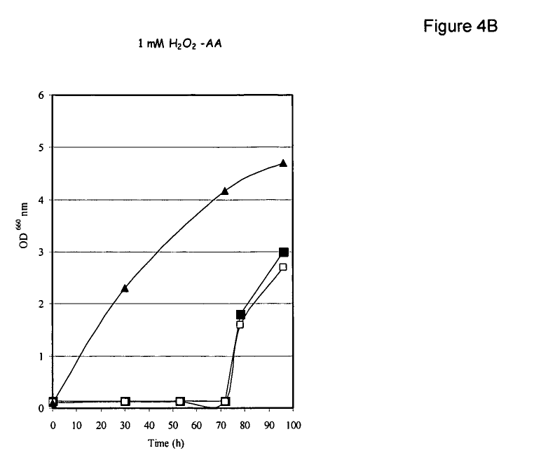

FIG. 4 shows that the growth defects of the YML007w yeast hosts can be rescued following expression of ALO, LDGH, ME, and MIP.

The following yeast strains have been analyzed:

BY4742

[symbol]

YML007w expressing ALO, LDGH and ME (□)

YML007w expressing ALO, LDGH, ME and MIP (▪)

FIG. 4A. The yeast strains were grown on mineral medium (2% glucose, 0.67% YNB) starting from an OD⁶⁶⁰ of 0.1 in presence of 0.8 mM of H₂O₂.

FIG. 4B. The yeast strains were grown on mineral medium (2% glucose, 0.67% YNB) starting from an OD⁶⁶⁰ of 0.1 in presence of 1.0 mM of H₂O₂.

The cloned genes allowed the rescue of the growth sensitivity similarly to that obtained by adding ascorbic acid n the culture medium (see FIG. 3).

It is interesting to note that the presence of MIP allowed a faster recovery when compared to yeast cells without it.

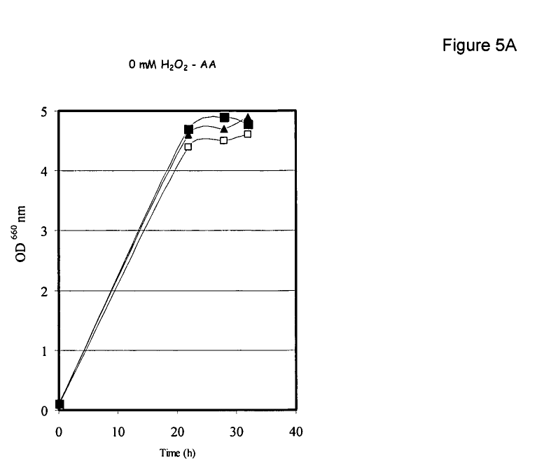

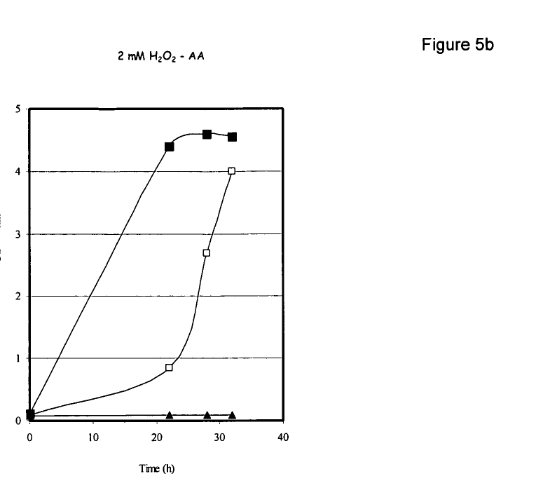

As a classical example of stress, we challenged wild type yeast cells with H₂O₂. As expected, wild type cells grow well in the absence of H₂O₂ (FIG. 5A), but the same yeast cells do not grow in the presence of the H₂O₂ (FIG. 5B). It is generally accepted that this external stressor leads to damage to DNA, damage to lipids, damage to proteins, and damage to membranes, among other subcellular structures, and ultimately leads to a loss of cell viability and cell integrity. Therefore, it is not surprising that the presence of this stressor leads to zero production, zero productivity and zero yield of the product (in this case, wild type yeast biomass), as shown in FIG. 5B.

By the transformation of wild type GRF yeast with (i) LGDH, ALO, and ME or (ii) LGDH, ALO, ME and MIP, the recombinant yeast produced ascorbic acid, as described above, whereas wild type yeasts do not naturally produce ascorbic acid. Surprisingly, the bioprocess based on these recombinant yeasts showed a high production, high productivity, and a high yield of the product, yeast biomass (FIG. 5B). Values for production, productivity, and yield are greater than 0.00 (values for the control strain).

FIG. 5 shows that the wild type GRF yeast strain is sensitive to fermentative stress conditions (stress condition induced by adding 2 mM of H2O2); surprisingly,the recombinant yeast strains producing ascorbic acid show a strong robustness. The following yeast strains have been analyzed: GRFc (closed triangle); GRF18U expressing ALO, LDGH and ME (open square); and GRF18U expressing ALO, LDGH, ME and MIP (closed square).

FIG. 5A. The yeast strains were grown on mineral medium (2% glucose, 0.67% YNB) starting from an OD660 of 0.1.

FIG. 5B. The yeast strains were grown on mineral medium (2% glucose, 0.67% YNB) starting from an OD660 of 0.1 in presence of 2.0 mM of H2O2. The wild type strain does not consume glucose.

All the strains used in this experiment bear the same auxotrophic complementation and the same antibiotic resistance cassettes (that are necessary for the expression of the different heterologous genes), so that it was possible to use the same media for all of them, either the ones expressing 3 or 4 heterologous genes or the wild type strain.

This experiment shows the two recombinant GRF yeast strains are more robust strains than wild type GRF yeast, and may therefore be more suitable for certain industrial processes. Though not to be bound by theory, we consider it likely the recombinant yeast are less sensitive to diverse stressors, possibly through both direct scavenging of reactive oxygen species (ROS) by ascorbic acid and interference by ascorbic acid with unwanted stress reactions like apoptosis, cell death, viability loss, and integrity loss, among others.

While the compositions and methods and yeast strains of this invention have been described in terms of particular embodiments, it will be apparent to those of skill in the art that variations may be applied without departing from the concept, spirit and scope of the invention.

### REFERENCES

The following references, to the extent that they provide exemplary procedural or other details supplementary to those set forth herein, are specifically incorporated herein by reference.

[1] Padh H. 1990, Cellular functions of ascorbic acid, Biochem. Cell Biol. 68, 1166-1173.

[2] U.S. Pat. No. 2,265,121

[3] Huh, W. K., Lee, B. H., Kim, S. T., Kim, Y. R., Rhie, G. E., Baek, Y. W., Hwang, C. S., Lee, S. J., Kang, S. O., 1998, D-Erythroascorbic acid is an important antioxidant molecule in S. cerevisiae, Mol. Microb. 30, 4, 895-903

[4] Wheeler, G. L., Jones, M. A., Smirnoff, N., 1998, The biosynthetic pathway of vitamin C in higher plants, Nature 393, 365-368

[5] Huh, W. K., Kim, S. T., Yang, K. S., Seok, Y. J., Hah, Y. C., Kang, S. O., 1994, Characterisation of D-arabinono-1,4-lactone oxidase from Candida albicans ATCC 10231, Eur. J. Biochem. 225, 1073-1079

[6] Kim, S. T., Huh, W. K., Kim, J. Y., Hwang, S. W., Kang, S. O., 1996, D-Arabinose dehydrogenase and biosynthesis of erythroascorbic acid in Candida albicans, BBA 1297, 1-8

[7] Kim, S. T., Huh, W. K., Lee, B. H., Kang, S. O., 1998, D-Arabinose dehydrogenase and its gene from Saccharomyces cerevisiae, BBA 1429, 29-39

[8] Roland, J. F., Cayle, T., Dinwoodie, R. C., Mehnert, D. W., 1986, Fermentation Production of Ascorbic Acid from L-Galactonic Substrate, U.S. Pat. No. 4,595,659

[9] Roland, J. F., Cayle, T., Dinwoodie, R. C., Mehnert, D. W., 1990, Bioconversion Production of Ascorbic Acid with L-Galactono-1,4-Oxidase, U.S. Pat. No. 4,916,068

[10] Lee, B. H., Huh, W. K., Kim, S. T., Lee, J. S., Kang, S. O., 1999, Bacterial Production of D-Erythroascorbic Acid and L-ascorbic acid through Functional Expression of Saccharomyces cerevisiae D-Arabinono-1,4-Lactone Oxidase in Escherichia coli, App. Env. Microb. 65, 10, 4685-4687

[11] Østergaard, J., Persiau, G., Davey, M. W., Bauw, G., Van Montagu, M., 1997, Isolation of a cDNA Coding for L-Galactono-γ-Lactone Dehydrogenase, an Enzyme involved in the Biosynthesis of Ascorbic Acid in Plants, J. Biol. Chem. 272, 48, 30009-30016

[12] Bauw, G. J. C., Davey, M. W., Østergaard, J., Van Montagu, M. C. E., 1998, Production of Ascorbic Acid in Plants, 1998, International Patent Application, WO98/50558

[13] Berry, A., Running, J., Severson, D. K., Burlingame, R. P., 1999, Vitamin C Production in Microorganisms and Plants, International Patent Application, WO99/64618

[14] Smirnoff, N., Wheeler, G., 1999, Plant Galactose Dehydrogenase, International Patent Application, WO99/33995

[15] Hancock, R. D., Galpin, J. R., and Viola, R. 2000, Biosynthesis of L-ascorbic acid (vitamin C) by Saccharomyces cerevisiae. FEMS Microbiol. Lett. 186, 245-250

[16] Nishikimi, M., Noguchi, E., Yagi, K., 1978, Occurrence in Yeast of L-Galactonolactone Oxidase Which is Similar to a Key Enzyme for Ascorbic Acid Biosynthesis in Animals, L-Gulonolactone Oxidase, Arch. Biochem. Biophys. 191, 2, 479-486

[17] Bleeg, H. S., Christensen, F., 1982, Biosynthesis of Ascorbate in Yeast, Purification of L-Galactono-1,4-lactone Oxidase with Properties Different from Mammalian L-Gulonolactone Oxidase, Eur. J. Biochem. 127, 391-96

[18] Sullivan, M. X., Clarke, H. C. N., 1955, A highly specific procedure for ascorbic acid, Assoc. Off. Agr. Chem. 38, 2, 514-518

[19] Lowry, O. H., Rosebrough, N. J., Farr, A. L., Randall, R. J., 1951, Protein Measurement with the Folin Phenol Reagent, J. Biol. Chem. 193, 265-275

[20] Minet, M., Dufour, M. E., Lacroute, F., 1992, Plant J., 2, 417-422

[21] Sambrook et al., Molecular Genetics: A Laboratory Manual, Cold Spring Harbor Laboratory Press

[22] Gietz, R. D. and Schiestl, R. H., 1996, Transforming Yeast with DNA, Methods in Mol. and Cell. Biol.

[23] Kreger-van Rij, N. J. W., “The Yeasts,” Vol. 1 of Biology of Yeasts, Ch. 2, A. H. Rose and J. S. Harrison, Eds. Academic Press, London, 1987.

[24] Brambilla, L., Bolzani, D., Compagno, C., Carrera, D., van Dijken, J. P., Pronk, J. T., Ranzi, B. M., Alberghina, L., Porro, D. 1999, NADH reoxidation does not control glycolytic flux during exposure of respiring Saccharomyces cerevisiae cultures to glucose excess, FEMS Microb. Lett. 171, 133-140

[25] Wésolowski-Louvel, M., Prior, C., Bornecque, D., Fukuhara, H. 1992, Rag-mutations involved in glucose metabolism in yeast: isolation and genetic characterization. Yeast 8, 711-719

[26] Kumar, M. 2000 Production of ascorbic acid using yeast, International patent application WO 00/34502

[27] Porro, D., et al., U.S. Pat. No. 6,630,330

What is claimed is:

1. A method of generating L-ascorbic acid, comprising:
a) obtaining a recombinant yeast functionally transformed with a coding region encoding a mannose epimerase (ME);
b) culturing the recombinant yeast in a medium comprising D-glucose, thereby forming L-ascorbic acid, and
c) isolating the L-ascorbic acid.

2. The method of claim 1, wherein the recombinant yeast is further functionally transformed with a coding region encoding a myoinositol phosphatase (MIP).

3. The method of claim 1, wherein the yeast belongs to the genus Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces.

4. The method of claim 3, wherein the yeast belongs to the species S. cerevisiae, K. lactis, or Z. bailii.

5. The method of claim 4, wherein the yeast is selected from S. cerevisiae strain GRF18U; S. cerevisiae strain W3031B, BY4742, and YML007w, K. lactis strain CBS2359, or Z. bailii strain ATCC 60483.

6. The method of claim 1, wherein the ME has at least about 95% identity with SEQ ID NO:1.

7. The method of claim 6, wherein the ME has at least about 98% identity with SEQ ID NO:1.

8. The method of claim 2, wherein the MIP has at least about 95% identity with SEQ ID NO:2.

9. The method of claim 8, wherein the MIP has at least about 98% identity with SEQ ID NO:2.

10. The method of claim 1, wherein the yeast is further functionally transformed with a coding region encoding an enzyme selected from L-galactose dehydrogenase (LGDH), L-galactono-1,4-lactone dehydrogenase (AGD), D-arabinose dehydrogenase (ARA), D-arabinono-1,4-lactone oxidase (ALO), or L-gulono-1,4-lactone oxidase (GLO).

11. The method of claim 1, wherein the coding region is linked to a promoter active in the yeast.

12. The method of claim 11, wherein the promoter is the S. cerevisiae triosephosphateisomerase (TPI) promoter.

13. The method of claim 1, wherein the isolating step comprises lysing the yeast.

14. The method of claim 13, wherein the isolating step further comprises centrifugation, filtration, microfiltration, ultrafiltration, nanofiltration, liquid-liquid extraction, crystallization, enzymatic treatment with nuclease or protease, or chromatography.

15. The method of claim 1, wherein the isolating step comprises chromatography, activated carbon, microfiltration, ultrafiltration, nanofiltration, liquid-liquid extraction, or crystallization.

16. A recombinant yeast, wherein the yeast is functionally transformed with a coding region encoding a mannose epimerase (ME).

17. The recombinant yeast of claim 16, wherein the recombinant yeast is further functionally transformed with a coding region encoding a myoinositol phosphatase (MIP).

18. The recombinant yeast of claim 16, wherein the ME has at least about 95% identity with SEQ ID NO:1.

19. The recombinant yeast of claim 17, wherein the MIP has at least about 95% identity with SEQ ID NO:2.

20. The recombinant yeast of claim 16, wherein the yeast is further functionally transformed with a coding region encoding an enzyme selected from L-galactose dehydrogenase (LGDH), L-galactono-1,4-lactone dehydrogenase (AGD), D-arabinose dehydrogenase (ARA), D-arabinono-1,4-lactone oxidase (ALO), or L-gulono-1,4-lactone oxidase (GLO).

21. A method of increasing the production, productivity, or yield of a product produced by a microorganism during fermentation, comprising:
functionally transforming the microorganism with a coding region encoding a mannose epimerase (ME).

22. The method of claim 21, further comprising functionally transforming the microorganism with a coding region encoding a myoinositol phosphatase (MIP).

23. The method of claim 21, wherein the microorganism is selected from the group consisting of bacteria, yeast, filamentous fungi, animal cells, and plant cells.

24. The method of claim 23, wherein the yeast belongs to the species S. cerevisiae, K. lactis, or Z. bailii.

25. The method of claim 24, wherein the yeast belongs to the strain S. cerevisiae GRF.


SEQUENCE LISTING (15 sequences)

SEQ ID NO: 1 - protein, 377 aa, Arabidopsis thaliana
MGTTNGTDYGAYTYKELEREQYWPSENLKISITGAGGFIASHIARRLKHEGHYVIASDWKKNEHMTEDMFCDEFHLVDLRVMENCLKVTEGVDHVFNLAADMGGMGFIQSNHSVIMYNNTMISFNMIEAARINGIKRFFYASSACIYPEFKQLETTNVSLKESDAWPAEPQDAYGLEKLATEELCKHYNKDFGIECRIGRFHNIYGPFGTWKGGREKAPAAFCRKAQTSTDRFEMWGDGLQTRSFTFIDECVEGVLRLTKSDFREPVNIGSDEMVSMNEMAEMVLSFEEKKLPIHHIPGPEGVRGRNSDNNLIKEKLGWAPNMRLKEGLRITYFWIKEQIEKEKAKGSDVSLYGSSKVVGTQAPVQLGSLRAADGKE

SEQ ID NO: 2 - protein, 271 aa, Arabidopsis thaliana
MADNDSLDQFLAAAIDAAKKAGQIIRKGFYETKHVEHKGQVDLVTETDKGCEELVFNHLKQLFPNHKFIGEETTAAFGVTELTDEPTWIVDPLDGTTNFVHGFPFVCVSIGLTIGKVPVVGVVYNPIMEELFTGVQGKGAFLNGKRIKVSAQSELLTALLVTEAGTKRDKATLDDTTNRINSLLTKVRSLRMSGSCALDLCGVACGRVDIFYELGFGGPWDIAAGIVIVKEAGGLIFDPSGKDLDITSQRIAASNASLKELFAEALRLTGA

SEQ ID NO: 3 - DNA, 1134 bp, Arabidopsis thaliana
atgggaactaccaatggaacagactatggagcatacacatacaaggagctagaaagagagcaatattggccatctgagaatctcaagatatcaataacaggagctggaggtttcattgcatctcacattgctcgtcgtttgaagcacgaaggtcattacgtgattgcttctgactggaaaaagaatgaacacatgactgaagacatgttctgtgatgagttccatcttgttgatcttagggttatggagaattgtctcaaagttactgaaggagttgatcatgtttttaacttagctgctgatatgggtggtatgggttttatccagagtaatcactctgtgattatgtataataatactatgattagtttcaatatgattgaggctgctaggatcaatgggattaagaggttcttttatgcttcgagtgcttgtatctatccagagtttaagcagttggagactactaatgtgagcttgaaggagtcagatgcttggcctgcagagcctcaagatgcttatggtttggagaagcttgctacggaggagttgtgtaagcattacaacaaagattttggtattgagtgtcgaattggaaggttccataacatttatggtccttttggaacatggaaaggtggaagggagaaggctccagctgctttctgtaggaaggctcagacttccactgataggtttgagatgtggggagatgggcttcagacccgttcttttacctttatcgatgagtgtgttgaaggtgtactcaggttgacaaaatcagatttccgtgagccggtgaacatcggaagcgatgagatggtgagcatgaatgagatggctgagatggttctcagctttgaggaaaagaagcttccaattcaccacattcctggcccggaaggtgttcgtggtcgtaactcagacaacaatctgatcaaagaaaagcttggttgggctcctaatatgagattgaaggaggggcttagaataacctacttctggataaaggaacagatcgagaaagagaaagcaaagggaagcgatgtgtcgctttacgggtcatcaaaggtggttggaactcaagcaccggttcagctaggctcactccgcgcggctgatggaaaagagtga

SEQ ID NO: 4 - DNA, 1028 bp, Arabidopsis thaliana
ctaggctcgagaagcttgtcgacgaattcagatatccatggcggacaatgattctctagatcagtttttggctgccgccattgatgccgctaaaaaagctggacagatcattcgtaaagggttttacgagactaaacatgttgaacacaaaggccaggtggatttggtgacagagactgataaaggatgtgaagaacttgtgtttaatcatctcaagcagctctttcccaatcacaagttcattggagaagaaactacagctgcatttggtgtgacagaactaactgacgaaccaacttggattgttgatcctcttgatggaacaaccaatttcgttcacgggttccctttcgtgtgtgtttccattggacttacgattggaaaagtccctgttgttggagttgtttataatcctattatggaagagctattcaccggtgtccaagggaaaggagcattcttgaatggaaagcgaatcaaagtgtcagctcaaagcgaacttttaaccgctttgctcgtgacagaggcgggtactaaacgagataaagctacattagacgatacaaccaacagaatcaacagtttgctaaccaaggtcaggtcccttaggatgagtggttcgtgtgcactggacctctgtggcgttgcgtgtggaagggttgatatcttctacgagctcggtttcggtggtccatgggacattgcagcaggaattgtgatcgtgaaagaagctggtggactcatctttgatccatccggtaaagatttggacataacatcgcagaggatcgcggcttcaaacgcttctctcaaggagttattcgctgaggcgttgcggcttacaggggcatgattatcacgaattctggatccgatacgtaacgcgtctgcagcatgcgtggtaccgagcttttccctatagtgagtcgtattagagcttggcgtaatcatggtcatagctgtttcctgtgtgaattgttatccgctcacatttcacacaacatacgagccggaagcataaagtgtaa

SEQ ID NO: 5 - DNA, 1581 bp, Saccharomyces cerevisiae
atgtctactatcccatttagaaagaactatgtgttcaaaaactgggccggaatttattctgcaaaaccagaacgttacttccaaccaagttcaattgatgaggttgtcgagttagtaaagagtgccaggctagctgaaaaaagcttagttactgttggttcgggccattctcctagtaacatgtgcgttactgatgaatggcttgttaacttagacagattggacaaagtacaaaagtttgttgaatatcctgagttacattatgccgatgtcacagttgatgccggtatgaggctttaccaattgaatgaatttttgggtgcgaaaggttactctatccaaaatttaggctctatctcagaacaaagtgttgctggcataatctctactggtagtcatggttcctcaccttatcacggtttgatttcttctcaatacgtaaacttgactattgttaatggtaagggcgaattgaagttcttggatgccgaaaacgatccagaagtctttaaagctgctttactttcagttggaaaaattggtatcattgtctctgctactatcagggttgttcccggcttcaatattaaatccactcaagaagtgattacttttgaaaaccttttgaagcaatgggataccctatggacttcatctgaatttatcagagtttggtggtacccttatactagaaaatgtgttctatggaggggtaacaaaactacagatgcccaaaatggtccagccaagtcatggtggggtaccaagctgggtagatttttctacgaaactctattatggatctctaccaaaatctatgcgccattaaccccatttgtggaaaagttcgttttcaacaggcaatatgggaaattggagaagagctctactggtgatgttaatgttaccgattctatcagcggatttaatatggactgtttgttttcacaatttgttgatgaatgggggtgccctatggataatggtttggaagtcttacgttcattggatcattctattgcgcaggctgccataaacaaagaattttatgtccacgtgcctatggaagtccgttgctcaaatactacattaccttctgaacccttggatactagcaagagaacaaacaccagtcccggtcccgtttatggcaatgtgtgccgcccattcctggataacacaccatcccattgcagatttgctccgttggaaaatgttaccaacagtcagttgacgttgtacataaatgctaccatttataggccgtttggctgtaatactccaattcataaatggtttaccctttttgaaaatactatgatggtagcgggaggtaagccacattgggccaagaacttcctaggctcaaccactctagctgctggaccagtgaaaaaggatactgattacgatgactttgaaatgagggggatggcattgaaggttgaagaatggtatggcgaggatttgaaaaagttccggaaaataagaaaggagcaagatcccgataatgtattcttggcaaacaaacagtgggctatcataaatggtattatagatcctagtgagttgtccgactag

SEQ ID NO: 6 - DNA, 2138 bp, Saccharomyces cerevisiae
cccatgtctactatcccatttagaaagaactatgtgttcaaaaactgggccggaatttattctgcaaaaccagaacgttacttccaaccaagttcaattgatgaggttgtcgagttagtaaagagtgccaggctagctgaaaaaagcttagttactgttggttcgggccattctcctagtaacatgtgcgttactgatgaatggcttgttaacttagacagattggacaaagtacaaaagtttgttgaatatcctgagttacattatgccgatgtcacagttgatgccggtatgaggctttaccaattgaatgaatttttgggtgcgaaaggttactctatccaaaatttaggctctatctcagaacaaagtgttgctggcataatctctactggtagtcatggttcctcaccttatcacggtttgatttcttctcaatacgtaaacttgactattgttaatggtaagggcgaattgaagttcttggatgccgaaaacgatccagaagtctttaaagctgctttactttcagttggaaaaatcggtatcattgtctctgctactatcagggttgttcccggcttcaatattaaatccactcaagaagtgattacttttgaaaaccttttgaagcaatgggataccctatggacttcatctgaatttatcagagtttggtggtacccttatactagaaaatgtgttctatggaggggtaacaaaactacagatgcccaaaatggtccagccaagtcatggtggggtaccaagctgggtagatttttctacgaaactctattatggatctctaccaaaatctatgcgccattaaccccatttgtggaaaagttcgttttcaacaggcaatacgggaaattggagaagagctctactggtgatgttaatgttaccgattctatcagcggatttaatatggactgtttgttttcacaatttgttgatgaatgggggtgccctatggataatggtttggaagtcttacgttcattggatcattctattgcgcaggctgccataaacaaagaattttatgtccacgtgcctatggaagtccgttgctcaaatactacattaccttctgaacccttggatactagcaagagaacaaacaccagtcccggtcccgtttatggcaatgtgtgccgcccattcctggataacacaccatcccattgcagatttgctccgttggaaaatgttaccaacagtcagttgacgttgtacataaatcctaccatttataggccgtttggctgtaatactccaattcataaatggtttaccctttttgaaaatactatgatggtagcgggaggtaagccacattgggccaagaacttcctaggctcaaccactctagctgctggaccagtgaaaaaggatactgattacgatgactttgaaatgagggggatggcattgaaggttgaagaatggtatggcgaggatttgaaaaagttccggaaaataagaaaggagcaagatcccgataatgtattcttggcaaacaaacagtgggctatcataaatggtattatagatcctagtgagttgtccgactagtctctttttgtctcaataatctctatattttactaaaaaagaatatatatatatatatttatatatagcagtgtgatgactgttcatgtacattctaataactattcctagctgcctatcaaagacttttttttgaattagagctttttagtaatcatgggacccttttttcttttcattatccttactatagtttttttttggaaaagccgaacgcggtaatgattggtcgtataagcaaaaacgaaacatcggcatggcataacgtagatcctatctacagggaagtttttagaaatcagatagaaatgtattttgagtgctgtatatattgcagtactttttttctctctaggatttaagtatgtttagtattaactcatatcacattttttctttgtaaaaagcaaccattcgcaacaatgtcgatagtagagacatgcatatcgtttgtttcgacaaatccgttttatccattttgtactggattgcttctgaattgtgtggttacaccgctttacttttggaaaacgcaaaatggtagaatcgtggtc

SEQ ID NO: 7 - DNA, 960 bp, Arabidopsis thaliana
atgacgaaaatagagcttcgagctttggggaacacagggcttaaggttagcgccgttggttttggtgcctctccgctcggaagtgtcttcggtccagtcgccgaagatgatgccgtcgccaccgtgcgcgaggctttccgtctcggtatcaacttcttcgacacctccccgtattatggaggaacactgtctgagaaaatgcttggtaagggactaaaggctttgcaagtccctagaagtgactacattgtggctactaagtgtggtagatataaagaaggttttgatttcagtgctgagagagtaagaaagagtattgacgagagcttggagaggcttcagcttgattatgttgacatacttcattgccatgacattgagttcgggtctcttgatcagattgtgagtgaaacaattcctgctcttcagaaactgaaacaagaggggaagacccggttcattggtatcactggtcttccgttagatattttcacttatgttcttgatcgagtgcctccagggactgtcgatgtgatattgtcatactgtcattacggcgttaatgattcgacgttgctggatttactaccttacttgaagagcaaaggtgtgggtgtgataagtgcttctccattagcaatgggcctccttacagaacaaggtcctcctgaatggcaccctgcttcccctgagctcaagtctgcaagcaaagccgcagttgctcactgcaaatcaaagggcaagaagatcacaaagttagctctgcaatacagtttagcaaacaaggagatttcgtcggtgttggttgggatgagctctgtctcacaggtagaagaaaatgttgcagcagttacagagcttgaaagtctggggatggatcaagaaactctgtctgaggttgaagctattctcgagcctgtaaagaatctgacatggccaagtggaatccatcagaactaa

SEQ ID NO: 8 - DNA, 22 bp, Artificial Sequence
  Forward PCR Primer for D-arabinono-1,4- lactone oxidase from S. cerevisiae
tttcaccatatgtctactatcc

SEQ ID NO: 9 - DNA, 22 bp, Artificial Sequence
  Reverse PCR Primer for D-arabinono-1,4- lactone oxidase from S. cerevisiae
aaggatcctagtcggacaactc

SEQ ID NO: 10 - DNA, 23 bp, Artificial Sequence
  Forward PCR Primer for L-galactose dehydrogenase from A. thaliana
atgacgaaaatagagcttcgagc

SEQ ID NO: 11 - DNA, 24 bp, Artificial Sequence
  Reverse PCR Primer for L-galactose dehydrogenase from  A. thaliana
ttagttctgatggattccacttgg

SEQ ID NO: 12 - DNA, 26 bp, Artificial sequence
  Forward PCR Primer for D-mannose-L-galactose epimerase from Arabidopsis thaliana
gcgccatgggaactaccaatggaaca

SEQ ID NO: 13 - DNA, 25 bp, Artificial sequence
  Reverse PCR Primer for D-mannose-L-galactose epimerase from  A. thaliana
gcgctcgagtcactcttttccatca

SEQ ID NO: 14 - DNA, 23 bp, Artificial sequence
  Forward PCR Primer for myoinositol phosphatase from A. thaliana
atccatggcggacaatgattctc

SEQ ID NO: 15 - DNA, 21 bp, Artificial sequence
  Reverse PCR Primer for myoinositol phosphatase from  A. thaliana
aatcatgcccctgtaagccgc